(19) United States
(12) Patent Application Publication
(10) Pub. No.: US 2006/0020316 A1
(43) Pub. Date: Jan. 26, 2006

(54) Implantable cardioversion and defibrillation system including intramural myocardial elecrtode

(75) Inventors: Gonzalo Martinez, Mendota Heights, MN (US); Natalia Trayanova, New Orleans, LA (US); Vinod Sharma, Blaine, MN (US)

Correspondence Address: MEDTRONIC, INC., 710 MEDTRONIC PARK, MINNEAPOLIS, MN, 55432-9924, US

(73) Assignee: Medtronic, Inc.

(21) Appl. No.: 10/859,641

(22) Filed: Jun. 3, 2004

(51) Int. Cl. A61N 1/05

(52) U.S. Cl. 607/122

(57) ABSTRACT

An implantable cardioverter defibrillation electrode system includes a cardioversion/defibrillation electrode mounted about an elongated lead body and an intramural electrode adapted for implantation within myocardial tissue.

### FIELD OF THE INVENTION

The present invention relates generally to implantable cardiac electrode systems and in particular to a cardioversion/defibrillation electrode system including an intramural electrode.

### BACKGROUND OF THE INVENTION

A major obstacle in achieving the first implantable defibrillation devices was reducing device size to a size acceptable for implantation. Large battery and capacitor requirements for delivering high-energy shock pulses required early devices to be relatively large. Most presently available implantable cardioverters and defibrillators (ICD's) are provided with an electrode system that includes one or more transvenously insertable leads, to be used alone or in conjunction with an additional subcutaneous electrode. Using truncated biphasic exponential waveforms for internal cardiac defibrillation via transvenously positioned electrodes has allowed defibrillation thresholds to be reduced to the point that device size is acceptable for pectoral implant. Defibrillator and transvenous electrode systems are illustrated in U.S. Pat. No. 4,953,551 issued to Mehra et al., U.S. Pat. No. 5,014,696 issued to Mehra and U.S. Pat. No. 5,261,400 issued to Bardy. Biphasic defibrillation waveforms are disclosed in the '551 patent issued to Mehra et al., and in U.S. Pat. No. 5,107,834 issued to Ideker et al., U.S. Pat. No. 4,821,723 issued to Baker, Jr. et and U.S. Pat. No. 4,850,357 issued to Bach.

Transvenously implantable electrodes in such systems typically take the form of an elongated coil, as disclosed in the above-cited references and may include electrodes located in the right ventricle, the coronary sinus or a cardiac vein, the superior vena cava/right atrium, or other locations relative to the myocardial tissue but remaining outside the myocardial tissue. The subcutaneous electrodes are typically implanted in the left pectoral or left axillary regions of the patient's body and may take the form of a separately implanted patch electrode or may comprise a portion of the housing of the associated implantable defibrillator.

Considerable progress has been made in reducing defibrillation thresholds in implantable systems, e.g., by introducing biphasic waveforms in place of monophasic waveforms and introducing transvenous electrode systems. The reduction in defibrillation energy requirements has allowed a reduction in implantable device size and increased device longevity, however room for improvement still exists. Further reduction in device size, increased device longevity, and potentially reducing pain perceived by a patient during shock delivery, continue to be motivating factors to improve implantable defibrillation systems by reducing defibrillation thresholds. Moreover, the efficacy rate of defibrillation therapy may be improved by reducing defibrillation thresholds, presumably by decreasing the number of patients with extremely high defibrillation thresholds.

In an effort to reduce the amount of energy required to effect defibrillation, numerous suggestions have been made with regard to multiple electrode systems. For example, sequential pulse multiple electrode systems are generally disclosed in U.S. Pat. No. 4,708,145 issued to Tacker et al., U.S. Pat. No. 4,727,877 issued to Kallok et al., U.S. Pat. No. 4,932,407 issued to Williams et al., and U.S. Pat. No. 5,163,427 issued to Keimel. An alternative approach to multiple electrode sequential pulse defibrillation is disclosed in U.S. Pat. No. 4,641,656 to Smits and also in the above-cited Williams patent. This defibrillation method may conveniently be referred to as multiple electrode, simultaneous pulse defibrillation and involves the delivery of defibrillation pulses simultaneously between two different pairs of electrodes. For example, one electrode pair may include a right ventricular electrode and a coronary sinus electrode, and the second electrode pair may include a right ventricular electrode and a subcutaneous patch electrode, with the right ventricular electrode serving as a common electrode to both electrode pairs. An alternative multiple electrode, simultaneous pulse system is disclosed in the previously referenced '551 patent issued to Mehra et al., employing right ventricular, superior vena cava and subcutaneous patch electrodes. Such multiple electrode systems generally employ transvenous electrodes wherein the electrodes used remain in the blood volume of a cardiac chamber or blood vessel and may be used in conjunction with an electrode in a subcutaneous location.

Pulse waveforms delivered either simultaneously or sequentially to defibrillation electrode systems may be monophasic (either of positive or negative polarity only), biphasic (having both a negative-going and positive-going pulse), or multiphasic (having two or more polarity reversals). Such waveforms thus include one or more pulses of negative and/or positive polarity that are typically truncated exponential pulses. These monophasic, biphasic, and multiphasic pulse waveforms are achieved by controlling the discharge of a capacitor or bank of capacitors during shock delivery. Other types of defibrillation therapy pulse regimes have been proposed for improving defibrillation efficacy or efficiency. Reference is made, for example, to U.S. Pat. No. 5,522,853 issued to Kroll and U.S. Pat. No. 6,415,179 issued to Pendekanti et al.

Other attempts at improving defibrillation therapy outcomes include delivering a pharmaceutical agent to the myocardial tissue to reduce defibrillation threshold or otherwise alter the electrophysiological state of the tissue. For example, the use of drugs in treating arrhythmias in conjunction with a defibrillation therapy is generally described in U.S. Pat. No. 6,571,125 issued to Thompson et al., and U.S. Pat. Appl. No. 2002/000071269 to Ideker et al.

One challenge in improving the effectiveness or efficiency of defibrillation therapies is that the underlying mechanism of defibrillation therapy is not fully understood. Even when using multiple electrode configurations, a relatively high-energy shock is still required in order to successfully defibrillate the heart. While ICDs have been shown to be highly effective in preventing sudden cardiac death, defibrillation therapy can still fail in some instances or require very high defibrillation energy in order to be successful. One mechanism that may explain why a defibrillation therapy may fail relates to virtual electrode polarizations within the myocardial mass produced by the defibrillation shock pulse. For shocks at or above the defibrillation threshold, the wave front emanating from the positively polarized areas rapidly excites negatively polarized areas post-shock, eliminating the post shock excitable gap and thus resulting in successful defibrillation. However, for shocks below defibrillation threshold, the wave front propagation elicited from the positive region travels relatively slowly through the negative region, allowing adjacent areas of shock-induced depolarization to recover; a reentrant activity, which is the basis of cardiac arrhythmias, can then ensue. Improved defibrillation systems that can manipulate the magnitude, location, and distribution of the virtual electrode polarization would improve defibrillation efficacy and reduce energy required for defibrillation.

### BRIEF DESCRIPTION OF THE DRAWINGS

The following drawings are illustrative of particular embodiments of the invention and therefore do not limit its scope, but are presented to assist in providing a proper understanding of the invention. The drawings are not to scale (unless so stated) and are intended for use in conjunction with the explanations in the following detailed description. The present invention will hereinafter be described in conjunction with the appended drawings, wherein like numerals denote like elements, and:

FIG. 1 is a plan view of a transvenous defibrillation lead according to one embodiment the present invention;

FIG. 2 is a plan view of an alternative embodiment of a transvenous defibrillation lead according to the present invention;

FIG. 3 is a plan view of yet another embodiment of the present invention;

FIG. 4 is schematic showing an embodiment of the present invention deployed within a patient's heart and coupled to an ICD;

FIG. 5 is another schematic showing another embodiment deployed within the heart;

FIG. 6 is a schematic illustration depicting a delivery tool used to deploy embodiments of the present invention; and

FIGS. 7A-B are detail views of alternate embodiments of the delivery tool shown in FIG. 6.

### DETAILED DESCRIPTION OF THE PREFERRED EMBODIMENTS

The following detailed description is exemplary in nature and is not intended to limit the scope, applicability, or configuration of the invention in any way. Rather, the following description provides a practical illustration for implementing exemplary embodiments of the invention.

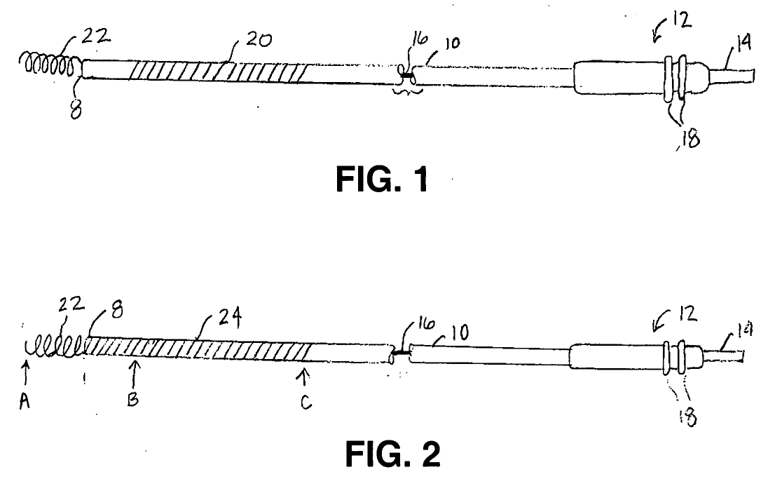

FIG. 1 is a plan view of a transvenous defibrillation lead according to one embodiment the present invention. The illustrated lead includes an elongated insulative lead body 10 which may be fabricated of polyurethane, silicone rubber or other biocompatible insulative material. Located at the proximal end of the lead is a connector assembly 12, which carries connector pin 14. Sealing rings 18 are provided to seal the connector assembly 12 within the connector block of an associated implantable cardioverter/defibrillator (ICD). FIG. 1 further illustrates an elongated cardioversion/defibrillation coil electrode 20 mounted on a distal portion of the lead body 10 and an intramural electrode 22 extending from a distal end 8 of lead body 10. According to the illustrated embodiment, cardioversion/defibrillation coil electrode 20 and intramural electrode 22 are electrically coupled via an electrical conductor 16 carried within insulative lead body 10 and extending between intramural electrode 22 and coil electrode 20 and proximally to connector pin 14 to allow electrical connection of coil electrode 20 and intramural coil 22 to a pulse generator included in an associated ICD.

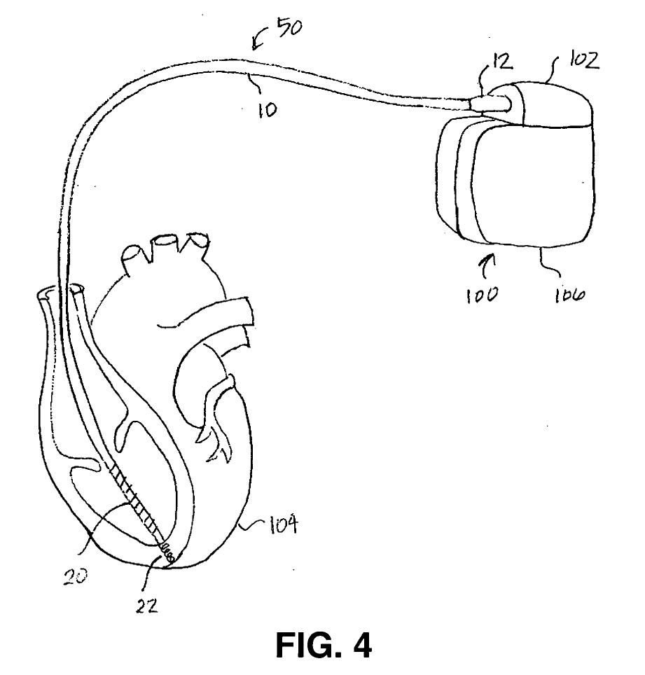

According to embodiments of the present invention, intramural electrode 22 is intended to be deployed within the myocardial tissue while the coil electrode 20 is intended for use as an extramural electrode, implanted within a body of a patient, but remaining outside the myocardial tissue. For example, the transvenous lead shown in FIG. 1 may be deployed in the right ventricle of the heart wherein the coil electrode 20 remains in the blood volume of the right ventricle and intramural electrode 22 is advanced into the myocardium of the right ventricular wall, as is depicted in FIG. 4. Intramural electrode 22 may be a near-transmural electrode, extending from the endocardial layer almost entirely through the myocardium to the epicardial layer, but without perforating the epicardial surface to avoid tamponade.

Coil electrode 20 may be fabricated as a conventional defibrillation coil electrode, such as a platinum-iridium coil electrode, as is well known in the art. According to embodiments of the present invention, intramural electrode 22 is fabricated to have greater current attenuation properties than coil electrode 20 so that during cardioversion/defibrillation shock delivery, relatively less current will flow through intramural electrode 22 than coil electrode 20 in order to prevent tissue damage. According to one embodiment, intramural electrode 22 includes a rectifier coating in order to achieve the desired current attenuation properties, for example electrode 22 may be fabricated in whole or in part of a valve metal such as tantalum, anodized and annealed to provide a thick, durable oxide coating.

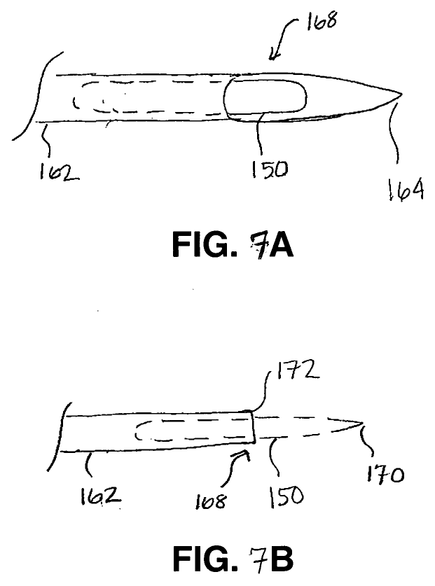

Intramural electrode 22 is shown in FIG. 1 in the form of a helical electrode that may be advanced into the myocardial tissue by rotating lead body 10. Such helical electrode designs are known for use in cardiac pacing, however, it is expected that the overall length of intramural electrode 22 may be longer than a typical pacing electrode so as to traverse a greater distance within the myocardial wall. Intramural electrode 22 may also take the form of an extendable/retractable helical electrode, known to those skilled in the art. Intramural electrode 22 may alternatively be embodied as any type of piercing electrode having a length and geometry that allows electrode 22 to be positioned intramurally, such as a fishhook, or stab-in type electrode. However, the design of intramural electrode 22 is not limited to a piercing-type electrode. Non-piercing intramural electrodes may be designed which are delivered to an intramural site using a piercing delivery tool, as will be described in conjunction with FIG. 7A.

FIG. 2 is a plan view of an alternative embodiment of a transvenous defibrillation lead according to the present invention. As illustrated in FIG. 2, intramural electrode 22 and coil electrode 24 are formed from one continuous structure having different current attenuation properties along its length; a portion of the structure extending from point A to point B is fabricated for an increased attenuation of delivered current to prevent damage to adjacent tissue in which intramural electrode 22 is implanted. The continuous structure, according to one embodiment, is formed from a tantalum wire, coated with platinum-iridium over a portion extending from point B to point C, and not provided with or stripped of the platinum-iridium coating between point A and point B. The exposed tantalum wire between point A and point B is anodized and annealed to provide a coating of tantalum oxide. According to another embodiment, a segment of portion A-B defined by intramural electrode 22 is fabricated to have even greater current attenuation properties than an adjacent segment of portion A-B defined by coil electrode 24. Methods for fabricating an electrode or portions of an electrode having increased attenuation of current are generally described in U.S. Pat. No. 5,848,031, to Martinez et al., incorporated herein by reference in its entirety.

During delivery of a cardioversion/defibrillation shock waveform, the portion extending from point A to point B displays an increased attenuation of current density, tending to shift current during the highest amplitude portion of the delivered waveform away from the tissue adjacent to distal lead end 8, reducing the likelihood that myocardial tissue will be damaged by delivery of the cardioversion/defibrillation shock.

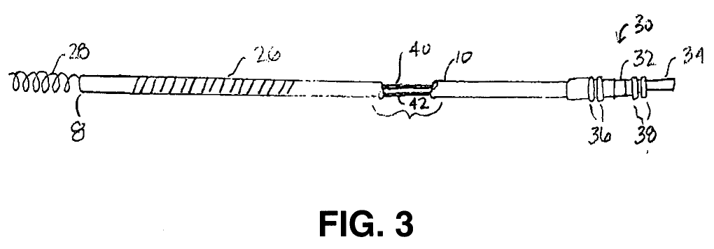

FIG. 3 is a plan view of yet another embodiment of the present invention. As illustrated in FIG. 3, a coil electrode 26 extending along a portion of lead body 10 is electrically isolated from an intramural electrode 28 extending from the distal end 8 of lead body 10. Each of intramural electrode 28 and coil electrode 26 are coupled to respective conductors 40 and 42 extending through lead body 10 to a proximal connector assembly 30. Connector assembly 30 is provided with a connector pin 34 and a connector ring 32, each of which is coupled to one of the respective conductors 40 and 42 extending to intramural electrode 28 or coil electrode 26. The conductors 40 and 42 are electrically isolated from each other within lead body 10. Sealing rings 36 are provided to seal the connector assembly 30 within the connector block of an associated ICD, and sealing rings 38 ensure electrical isolation between connector pin 34 and connector ring 32.

Connector pin 34 and connector ring 32 may be coupled to separate pulse generating output circuitry of an associated ICD such that cardioversion/defibrillation shock waveforms of differing shapes and energies may be delivered to intramural electrode 28 and coil electrode 26. Furthermore, a shock waveform delivered to intramural electrode 28 may be delivered before, simultaneously or sequentially with variable delay following a waveform delivered to coil electrode 26. By delivering a shock waveform via intramural electrode 28 at a time somewhat later than the shock pulse delivered to coil electrode 26, the re-entrant circuit elimination effect of direct energy delivery to the myocardial tissue may be optimized. In one application, a defibrillation waveform may be delivered first to coil electrode 26 and a second, relatively lower, defibrillation shock waveform may be delivered to intramural electrode 28 at a desired time interval after the delivery of the first shock waveform. In order to prevent myocardial tissue damage, intramural electrode 28 may include a rectifier coating or other current attenuation properties as described previously.

A biphasic defibrillation waveform is commonly delivered by commercially available ICD's. The present invention may be used in conjunction with any known cardioversion/defibrillation shock waveforms such as monophasic, biphasic, or multiphasic waveforms. Furthermore, the shock waveforms selected for delivery by an extramural cardioversion/defibrillation electrode and for delivery by an intramural electrode may be different. The type of waveform delivered by intramural electrode 28 may be selected in order to optimize the defibrillation threshold and the prevention or elimination of phase singularities.

FIG. 4 is schematic showing an embodiment of the present invention deployed within a patient's heart and coupled to an ICD. A lead 50 shown in FIG. 4 corresponds to the lead shown previously in FIG. 1, however, any of the leads shown in FIGS. 1 through 3 may be similarly deployed. FIG. 4 illustrates connector assembly 12 inserted in connector block 102 of the ICD 100, and a distal portion of the lead 50 extending within a right ventricle of the heart 104, with intramural electrode 22 located in the right ventricular apex.

The delivery of current directly to the myocardial tissue in the vicinity of intramural electrode 22 is intended to provide a region of shock-induced refractoriness that might act as a block to wavefront propagation in the region of the apex. Based on studies of the mechanisms of defibrillation therapy using bidomain modeling, there is a high probability that the post-shock reentry will include a figure-of-eight circuit with an isthmus at the apex, rendering the latter a target for reentry elimination. Since the strong positive surface polarization created by an electrode in the blood pool extends only a few cell layers beneath the endocardium, an electrode extended to within the apical myocardium is more likely to create strong positive polarization within the bulk of the tissue there.

ICD 100 contains within housing 106 one or more high voltage capacitors defining a capacitor bank having a first pole coupled to a circuit ground and a second pole adapted to be coupled to a high voltage charging circuit, such that on completion of charging, the capacitor bank retains a voltage of up to plus 750 to plus 800 volts, relative to circuit ground. Output control circuitry controls the delivery of a cardioversion/defibrillation waveform during capacitor discharge.

During shock delivery, current density is shifted away from intramural electrode 22, to reduce the likelihood of tissue damage in the right ventricular apex. However, delivery of active current directly to the myocardium, particularly in the region of the ventricular apex, is expected to effectively eliminate reentrant circuits that may occur following shock delivery, which might otherwise give rise to the genesis of a new arrhythmia. Other locations for implanting an active intramural electrode, however, may also be found to be effective in lowering defibrillation thresholds and/or preventing or extinguishing re-entrant circuits. A lead having an extramural cardioversion/defibrillation electrode and an intramural electrode may alternatively be positioned in a cardiac vein via the coronary sinus. Furthermore, it is recognized that multiple leads carrying active intramural electrodes may be deployed at various myocardial locations.

According to an alternate embodiment of the present invention, electrode 22 is not electrically coupled to electrode 20 and serves as an intramural passive electrode preferably formed from an implantable grade solid insulating material, such as silicone polyurethane, or a fluoropolymer. but semiconductors, ceramics, glasses, oxides, metals and metal alloys can also be used. As a passive electrode, electrode 22 forms a discontinuity in the myocardial structure, thereby giving rise to polarization of the tissue bordering electrode 22 during cardioversion/defibrillation shock delivery via coil electrode 20. Thus, as a passive intramural electrode, electrode 22 does not actively deliver current to the myocardial tissue but rather acts as a “virtual source” by causing polarization of the tissue in its vicinity. In order to effectively form a discontinuity in the myocardial structure, it is expected that electrode 22 preferably be formed from a solid insulating material, however, passive electrodes formed from other semiconductor or conductive materials may be found to be effective in reducing defibrillation thresholds as well. Passive elements can also be formed selectively by electromagnetic radiation, chemical, electrical or surgical methods that form regions of significantly lower conductivity than that of the tissue (scar or calcified tissue).

By forming one or more discontinuities in the myocardial structure by deploying one or more passive electrodes, a reduced defibrillation threshold is anticipated, thereby improving the efficacy of defibrillation therapies. The dimensions and positioning of passive electrodes is a factor contributing to their effectiveness in reducing defibrillation thresholds. The propagation of an electrical wave front through myocardial tissue is characterized by a wavelength, which is the product of the myocardial conduction velocity and the action potential duration (or effective refractory period); the length/perimeter of a passive electrode is preferably less than one wavelength for if the length/perimeter is greater than or equal to one wavelength, the passive electrode may provide a substrate for re-entrant currents. The maximum allowable length/perimeter of a passive electrode based on the wavelength concept is computed to be on order of about 10 to 15 cm. However, in order to facilitate implantation of the passive electrode in the myocardium, the passive electrode may be considerably shorter, on the order of a few centimeters or less but greater than a minimum effective length. A minimum effective length is expected to exist in that a passive intramural electrode shorter than the minimum effective length will not act as a significant virtual source and cause the desired polarization effect.

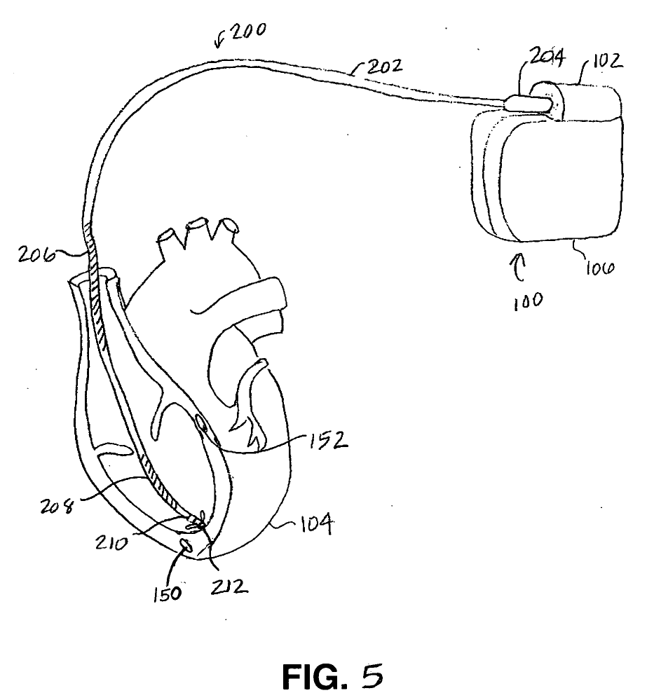

Both the location of a passive electrode with respect to the heart anatomy and the orientation of the passive electrode relative to myocardial fiber direction may be important factors in optimizing the effectiveness of the passive electrode in reducing defibrillation thresholds. An effective location is expected to be near the ventricular apex, as illustrated in FIG. 4, and an effective orientation may be one approximately parallel (as opposed to perpendicular) to myocardial fiber direction. However, optimal positioning of a passive electrode may depend upon the positioning of the active cardioversion/defibrillation electrodes used to deliver a shock waveform, inter-individual anatomical differences, and possibly the region of the heart giving rise to a re-entrant arrhythmia. Deployment of multiple passive electrodes, as illustrated in FIG. 5, may have greater effectiveness than deployment of a single passive electrode.

FIG. 5 is another schematic showing an embodiment including multiple passive electrodes deployed within the heart. FIG. 5 illustrates a first passive intramural electrodes 150 deployed in the region of the ventricular apex of heart 104 and a second passive intramural electrode 152 deployed in the region of the base of heart 104; electrodes 150, 152 are not carried by a lead having been deployed within the myocardial tissue using a delivery tool.

FIG. 5 further illustrates a lead 200 as a conventional transvenous cardioversion/defibrillation lead including a tip electrode 212 and a ring electrode 210 for pacing and sensing functions in addition to the right ventricular coil electrode 208 and a superior vena cava coil electrode 206. According to one embodiment, lead 200 includes a quadripolar in-line connector assembly 204 shown inserted in connector block 102 of ICD 100. Insulated conductors extending within lead body 202 to coil electrodes 206 and 208 are coupled via connector assembly 204 to high-voltage output circuitry contained within ICD 100. Likewise, respective insulated conductors carried by lead body 202 to tip electrode 212 and ring electrode 210 are coupled via connector assembly 204 to pacing output circuitry and sense amplifier circuitry contained within ICD 100.

Passive intramural electrodes may be used in conjunction with any available cardioversion/defibrillation leads for delivering the cardioversion/defibrillation shock waveform. However, passive intramural electrodes are not limited to use with lead-based high-voltage electrode systems. Passive intramural electrodes may be deployed for use with leadless electrode systems such as the subcutaneous implantable cardioverter-defibrillator generally disclosed in U.S. Pat. No. 6,647,292, issued to Bardy et al.

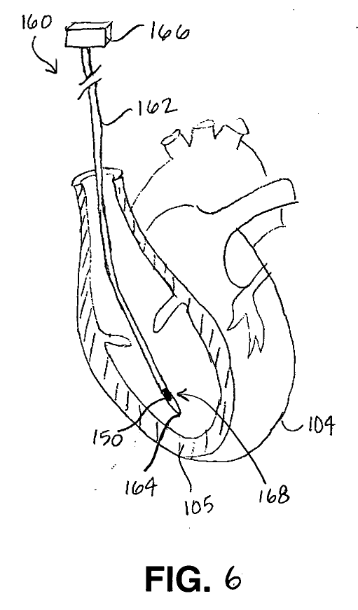

FIG. 6 is a schematic illustration depicting a delivery tool 160 used to deploy embodiments of the present invention. FIG. 6 illustrates delivery tool 160 as a catheter, hollow needle-like device, or other delivery device including an elongated, flexible body 162 capable of retaining a passive intramural electrode 150 and advancing the passive intramural electrode along a transvenous pathway to a desired myocardial site. According to one embodiment, delivery tool body 162 includes a relatively sharp, piercing distal tip 164, as shown in detail in FIG. 7A, such that, upon reaching the desired myocardial site, the distal tip 164 may be inserted into the myocardial tissue. FIG. 6 further illustrates delivery tool 160 including a proximal actuator 166 for causing the release of passive intramural electrode 150 from the distal end 168 of delivery tool 160. Actuator 166 may be designed as a mechanical, electrical, or thermal actuator which either forces passive electrode 150 out of distal end 164 of delivery tool 160 and/or causes the inner diameter of distal end 164 of delivery tool 160 to widen slightly to release electrode 150. Delivery tool 160 may then be removed from the myocardium 105 and heart 104 such that passive intramural electrode 150 remains within the myocardium 105 at the desired implant site. The passive intramural electrode 150 may be provided with a relatively blunt geometry such that after being positioned in the myocardium 105, the passive intramural electrode 150 does not perforate or migrate through the myocardium 105.

According to an alternate embodiment, and as shown in detail in FIG. 7B, the delivery tool 160 of FIG. 6 includes a relatively blunt, non-piercing tip 172 and the passive intramural electrode 150 includes a relatively sharp, piercing geometry, e.g. a sharpened tip 170 as shown in FIG. 7B. The delivery tool 160 may be used to advance the passive intramural electrode 150 to a desired myocardial site, and then to press the passive electrode against the myocardium 105 so as to pierce the myocardial wall and then to advance the passive electrode into the myocardium 105, using proximal actuating mechanism 166, while the delivery tool tip 164 remains outside or flush with the myocardial surface.

One medical device delivery system that may be adapted for deploying a passive intramural electrode is generally described in U.S. patent application Ser. No. 10/252,243 (Atty. Docket P-10213) to Geske, et al., hereby incorporated herein by reference in its entirety. The medical device delivery system includes a closable collet near the distal end of a guide body for engaging a medical device. The medical device is released by retracting a closing member to open the closable collet. The closable collet may be provided with a relatively blunt or sharpened, hypodermic needle-like tip.

In the foregoing detailed description, the invention has been described with reference to specific embodiments. However, it may be appreciated that various modifications and changes can be made without departing from the scope of the invention as set forth in the appended claims.

What is claimed is:

1. An implantable cardioverter defibrillation electrode system, comprising:
an elongated lead body;
a cardioversion/defibrillation electrode mounted about the lead body; and
an active intramural electrode adapted for implantation within myocardial tissue;
wherein the active intramural electrode displays greater current attenuation properties than the cardioversion/defibrillation electrode when a cardioversion/defibrillation shock is delivered via the cardioversion/defibrillation electrode and the active intramural electrode.

2. The system of claim 1, wherein the active intramural electrode extends from a distal end of the lead body.

3. The system of claim 1, wherein the active intramural electrode is electrically coupled to the cardioversion/defibrillation electrode.

4. The system of claim 3, wherein the active intramural electrode and the cardioversion/defibrillation electrode are a continuous structure.

5. The system of claim 1, wherein the active intramural electrode is electrically isolated from the cardioversion/defibrillation electrode.

6. The system of claim 1, wherein the active intramural electrode includes a rectifier coating.

7. The system of claim 1, wherein the active intramural electrode is formed of a valve metal and includes an oxide coating.

8. The system of claim 7, wherein the valve metal comprises tantalum.

9. The system of claim 4, wherein the continuous structure is formed of a helically wound tantalum wire including a platinum-iridium coating extending along a portion of the cardioversion/defibrillation electrode.

10. The system of claim 1, further comprising an intramural passive electrode adapted for implantation within myocardial tissue.

11. The system of claim 10, wherein the passive intramural electrode is formed from an insulating material.

12. The system of claim 1, further comprising a plurality of intramural passive electrodes adapted for implantation within myocardial tissue.

13. A method for delivering cardioversion/defibrillation therapy, the method comprising the steps of:
implanting an intramural electrode within myocardial tissue;
positioning a cardioversion/defibrillation electrode mounted about a lead body within a chamber of a heart;
delivering a cardioversion/defibrillation shock via the intramural electrode; and
delivering a cardioversion/defibrillation shock via the cardioversion/defibrillation electrode;
wherein the intramural electrode displays greater current attenuation properties than the cardioversion/defibrillation electrode.

14. The method of claim 13, wherein the intramural electrode extends from a distal end of the lead body.

15. The method of claim 13, wherein the intramural electrode is electrically coupled to the cardioversion/defibrillation electrode.

16. The method of claim 15, wherein the intramural electrode and the cardioversion/defibrillation electrode are a continuous structure.

17. The method of claim 13, wherein the intramural electrode is electrically isolated from the cardioversion/defibrillation electrode.

18. The method of claim 17, wherein a waveform of the cardioversion/defibrillation shock delivered via the transmural electrode is different from the waveform of the cardioversion/defibrillation shock delivered via the cardioversion/defibrillation electrode.

19. The method of claim 17, wherein a time delay exists between the cardioversion/defibrillation shock delivered via the transmural electrode and the cardioversion/defibrillation shock delivered via the cardioversion/defibrillation electrode.

20. The method of claim 13, further comprising the step of implanting a passive transmural electrode within myocardial tissue.

21. The method of claim 13, wherein the myocardial tissue is located in proximity to an apex of the heart.

22. An implantable cardioverter defibrillation electrode system, comprising:
an elongated lead body;
a cardioversion/defibrillation electrode mounted about the lead body; and
a passive intramural electrode adapted for implantation within myocardial tissue.

23. The system of claim 22, wherein the passive intramural electrode extends from a distal end of the lead body.

24. The system of claim 22, wherein the passive intramural electrode is formed from an insulating material.

25. The system of claim 22, wherein the passive intramural electrode is formed from a conductive material.
